(19) United States
(12) Patent Application Publication
(10) Pub. No.: US 2012/0029319 A1
(43) Pub. Date: Feb. 2, 2012

(54) Polysomnography method with remote administration

(76) Inventors: Ralph La Guardia, Mansfield Center, CT (US); Carl J. Tyler, Amston, CT (US)

(21) Appl. No.: 13/067,696

(22) Filed: Jun. 21, 2011

Related U.S. Application Data
(63) Continuation-in-part of application No. 12/805,365, filed on Jul. 27, 2010.

(51) Int. Cl. A61B 5/00

(52) U.S. Cl. 600/301

(57) ABSTRACT

A method wherein Type I (i.e., attended) polysomnography may be conducted at a distance from a patient by combining Internet-enabled remote access technologies, audioconferencing, and/or videoconferencing. The study is “virtually” attended by a polysomnography professional at a site removed from the patient whereby the polysomnography professional is able to control the equipment at the patient site and administer multiple Type I, virtually attended polysomnography studies.

### RELATED APPLICATIONS

This is a Continuation-In-Part of U.S. patent application Ser. No. 12/805,365 filed Jul. 27, 2010.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

None.

### FIELD OF THE INVENTION

The present invention generally relates to polysomnography for quantifying and diagnosing sleep disorders, for example, sleep apnea.

### BACKGROUND OF THE INVENTION

Polysomnography (PSG), also known as a sleep study, is a multi-parametric test used in the study of sleep and as a diagnostic tool in sleep medicine. It is a comprehensive recording of the biophysiological changes that occur during sleep. The procedure monitors many body functions including brain (EEG), eye movements (EOG), muscle activity or skeletal muscle activation (EMG) and heart rhythm (ECG) during sleep. Subsequent identification of the sleep apnea disorder in the 1970s, respiratory airflow and respiratory effort indicators, and peripheral pulse oximetry were included to facilitate diagnosis.

Polysomnography is used to diagnose or rule out many types of sleep disorders, including narcolepsy, periodic limb movement disorder, REM (rapid eye movement) behavior disorder, various parasomnia, and sleep apnea. Diagnosis of various sleep disorders is important not only for that specific disorder but also because of the statistical relationship of the disorder and other diseases, for example sleep apnea and heart attacks.

Obstructive sleep apnea is one of the most common disorders in the United States and is known to be a major cause of cardiovascular morbidity including heart attack and stroke. The development of a diagnostic system allowing an easy and simplified diagnosis of sleep apnea could prevent hundreds of thousands of annual excess deaths, heart attacks, and strokes. Once sleep apnea is diagnosed it is relatively easily treated. Because the disease is so common and results in other symptoms such as excessive daytime sleepiness, headaches, and decreased concentration it is imperative that an inexpensive diagnostic be developed.

The cost of diagnosing sleep apnea using a traditional approach of complex laboratory testing for every patient having the disease in the Unites States would be prohibitive. Typically, the procedure records a minimum of eleven channels requiring a minimum of 22 wire attachments to the patient. Two channels are for the EEG, one or two channels measure airflow, one channel measures chin movements, one or more channels measure leg movements, two channels detect eye movement, one channel for heart rate and rhythm, one channel for oxygen saturation and one channel each for the belts which measure chest wall movement and upper abdominal wall movement. These telemetrics converge into a central unit, which in turn is connected to a computer system for recording, storing and displaying the data. Additionally, most facilities include a video camera in the room so the technician can observe the patient from an adjacent room.

PSG studies are most commonly conducted in a sleep laboratory in a medical facility, such as a hospital. However, as the populace and their medical providers have become more aware of this procedure and its diagnostic value, increasing demand and the relatively limited number of in-patient sleep laboratories has resulted in long waiting times before patients can be accommodated. Additionally, the procedure is expensive when conducted in a hospital or specialized setting. Hence, home sleep testing solves a number of existing problems by using a portable device attached to the patient in his or her home. Such home sleep testing also provides a less expensive screening technology for sleep disorder detection. The advantages include, the ability to record data in the patient's most natural sleep environment (i.e., as compared to the laboratory setting of a traditional PSG study), greater availability (decreased wait time), decreased cost (usually <$1000 per study), and centralization of data analysis (hence decreased variability). However, disadvantages to such home sleep testing include inability to diagnose other nonbreathing-related sleep disorders and the potential for a greater number of invalid studies because testing is performed in unattended surroundings.

The federal Center for Medicare and Medicaid Services (CMS) defines a conventional PSG study performed in a sleep laboratory in a medical facility as a Type I procedure. These “attended” PSG studies (i.e., performed with the oversight of medically trained personnel, for example a sleep technologist) include full sleep staging whereby transition through the sleep stages can be monitored. The full range of modalities discussed above are generally utilized and the procedure must include at least:
EEG
EOG
ECG/Heart rate
Chin EMG
Limb EMG
Respiratory effort at thorax and abdomen
Air Flow from nasal canula thermistor and/or X-Flow
Pulse Oximetry
Additional channels for CPAP/BiPap levels, CO2, pH, pressure, etc.
   Type II, III, and IV studies are the previously described “home sleep test” (HST) studies, with each type differing in the number and/or type of modalities used. The patient is either instructed in applying the varying equipment of the apparatuses to him or herself prior to attempting sleep or a technician visits the home in order to connect his equipment to the patient. In contrast to the Type I study, the equipment operates autonomously and no medical personnel are present as the study progresses. As defined by CMS, a Type II study entails at least:
EEG
EOG
ECG/Heart rate
EMG
Airflow
Respiratory effort
Oxygen saturation
   A Type III study requires at least:
2 respiratory movement/airflow
ECG/Heart rate
Oxygen saturation
   Finally, a Type IV study includes a portable monitor having at least three channels. A Type IV device must allow channels that allow direct calculation of an Apnea and Hypopnea Index (AHI) or Respiratory Disturbance Index (RDI) as the result of measuring airflow or thoracoabdominal movement.

As noted supra, the information potentially available upon which a medical diagnosis must be based becomes progressively less comprehensive as the numerical value given to the study increases. While the most comprehensive information, and presumably the most precise diagnosis, is putatively available via a Type I study, the comparative absence of information in Type II-IV studies must be balanced with the likelihood of obtaining invalid results caused by patients' lack of familiarity with their surroundings, and the resulting sleep discomfort that may occur during a Type I study. While significantly more data is available during a Type I study, the patient is less likely to experience a typical night of sleep due to his unfamiliar surroundings. Conversely, Types II-IV studies are conducted in the privacy of one's home and provide familiar surroundings but generally do not provide the degree of information available in a Type I study.

What is needed, therefore, is a process and associated equipment whereby the comprehensive testing provided by a Type I PSG may be completed in the home. Previous attempts at providing Type I PSG studies in the home where the operator performs the testing from a remote site have been unsuccessful. Because Type I polysomnography typically includes direct visualization of the patient's respiratory activity, the inability to provide high quality, preferably bi-directional video has been a limiting factor in previous attempts. Such video can be important to record the patient's internal throat tissue during the awake and sleep stages so that if surgery is necessary it can be precisely undertaken with regard to the specific patient. It is known to use the remote desktop application Virtual Network Computing (VNC) to permit an operator at a site remote from the patient to access hardware at the patient's bedside.¹ In this previous attempt, a Microsoft Windows® based polysomnography platform was used and a common video camera was simply added so that the remote operator could view the camera's self view on the Windows® endpoint from his or her remote site. All the data was necessarily transmitted via the VNC remote desktop protocol with the result that video quality was limited by the bandwidth used by VNC. Video frame rates via this method are typically on the order of 2-4 frames per minute and therefore unusable with regard to conducting Type I PSG studies. Moreover, again due primarily to bandwidth limitations, audio could not be transmitted. Instead, the operator called the testing subject on his/her home telephone. Finally, the inherent insecurity of the VNC application raises concerns regarding compliance with the Health Insurance Portability and Accountability Act of 1996 (HIPAA) regulations under which all health care providers must operate. ¹ Kayyali H A., Weimer S., Frederick C., Martin C., Basa D., Juguilon J. A., et al. Remotely attended home monitoring of sleep disorders. Telemed J E Health. 2008;14:371-4.

A number of prior art patents and publications exist for remote PSG studies. U.S. Pat. No. 6,425,861 to Haberland et al. issued Jul. 30, 2002 describes a polysomnography testing apparatus incorporating a communications network It uses the ethernet communications protocol commonly used on local area networks (LAN) to facilitate separation of the instrumentation attached to the patient and the corresponding computing equipment receiving the data. In this manner the hardware in the patient's room is minimized such that the testing facility may be configured to more closely resemble a bedroom in a home. Additionally, because the polysomnography operator no longer need be present nearby during testing, the operator may be positioned at a more central location that allows him or her to administer testing to multiple patients simultaneously. The '861 patent does not contemplate remote control of the testing apparatus over extended distances and, in fact, the ethernet protocol is limited to distances on the order of approximately 150 meters. In other words, it may be used to administer multiple sleep studies within a single building or facility but cannot accommodate significant distances, for example, the distance between a patient's home and the central facility from which testing is being administered.

U.S. Pat. No. 7,674,230 to Reisfeld issued Mar. 9, 2010 describes conducting polysomnographic-type studies using computer aided interpretation of data from photoplethysmograph signals outputted by a pulse oximeter. The apparatus uses a limited stream of data to deduce information such as the presence of premature ventricular contractions and Cheyne-Stokes type respiration that may be indicative of sleep disorders. Type I PSG studies cannot be conducted using this specialized device. It utilizes a “console” coupled via a communications network to a “diagnostic processor” which performs the necessary analyses. Data flows unidirectionally to the “processor” so that the device cannot operate interactively with the patient. The operator is limited in the scope of administration and cannot, for example, communicate with the patient in the event that testing goes awry.

U.S. Patent Publication Number 2007/0130287 to Kumar et al. published Jun. 7, 2007 discloses an Internet browser based architecture for the transmission of physiological data over a wide area network (WAN), The system may also be used in conjunction with voice and videoconferencing. The publication discloses the use of custom browser plug-ins and applets in order to support a variety of medical devices. It is a point-to-point implementation whereby the medical device operator is in so-called “virtual” contact with the patient. It does not support the presence of a third party; for example, when the medical device operator encounters an exigency requiring intervention of a treating physician. Under these circumstances, what is needed is a mechanism by which the medical device operator can initiate communications, for example a videoconference, with a third party, such as a treating physician.

U.S. Patent Publication Number 2006/0135854 to McDonough et al. published Jun. 22, 2006 shows a system for remotely accessing polysomnography information securely over the Internet. It is primarily a storage solution and does not contemplate live, real-time interaction with the patient.

U.S. Patent Publication Number 2008/0033304 to Dalal et al. published Feb. 7, 2008 discloses an implantable device and method for diagnosing sleep disorders in which an implantable medical device or external physiological monitor may be coupled to an external device via telecommunications or computer network. The device may be interfaced with a continuous positive airway pressure (CPAP) device. Voice and/or video communications with the patient as well as internal video of the patient are not contemplated.

### SUMMARY OF THE INVENTION

The present invention utilizes existing polysomnography equipment in conjunction with existing remote access technologies and existing videoconferencing technologies to provide the administration of Type I PSG studies in the home as well as diagnostic video of the patient's internal soft tissues. In a preferred embodiment, polysomnography equipment and adjunct videoconferencing and audioconferencing equipment is utilized at a remote patient site. Remote access technology is utilized over a communications network, for example the Internet, to thereby facilitate remote administration of the aforementioned polysomnography equipment by a polysomnography professional located at another site removed from the patient site. The remote access technology facilitates bi-directional video and/or voice communications via an independent, unconstrained data stream. Type I PSG studies can be conducted in the home, in the most accurate manner and including high quality video which is necessary for visualization of the testing subjects respiratory activity. Moreover, Type I PSG procedure may now be administered to multiple patients by a single PSG professional, thereby reducing the cost of providing the procedure. It is further contemplated that the Type I PSG procedure may encompass the inclusion of ambulatory EEG with a video component.

It is an object of the invention to administer Type I PSG studies remotely;

It is yet another object of this invention to facilitate the administration of comprehensive, Type I PSG studies in the home;

It is another object of the invention to accurately diagnose sleep disorders by facilitating the administration of Type I PSG studies in the home;

It is still another object of this invention to accurately diagnose sleep apnea by facilitating the administration of Type I PSG studies in the home;

It is an object of this invention to record comprehensive PSG study data in the patient's most natural sleep environment;

It is yet another object of this invention to record accurate Type I PSG data in the patient's most natural sleep environment;

It is still another object of this invention to increase the availability of PSG studies to a wider number of patients;

It is an object of this invention to reduce the cost of administering Type I PSG studies;

It is a further object of this invention to reduce the number of invalid or incomplete PSG studies currently being conducted in the home; and,

It is an object of this invention to facilitate the use of HST PSG studies to diagnosis both breathing related sleep disorders, for example sleep apnea, and non-breathing related sleep disorders

The invention will be better understood and objects other than those set forth above will become apparent when consideration is given to the following detailed description thereof. Such description makes reference to the annexed drawings herein.

### BRIEF DESCRIPTION OF THE DRAWINGS

FIG. 1 is a schematic representation of the novel method of the invention;

FIG. 2 is a flowchart of a preferred embodiment of the invention; and,

FIG. 3 is a schematic representation of an interface with a sound generation device, camera, and a computer.

### DETAILED DESCRIPTION OF THE INVENTION

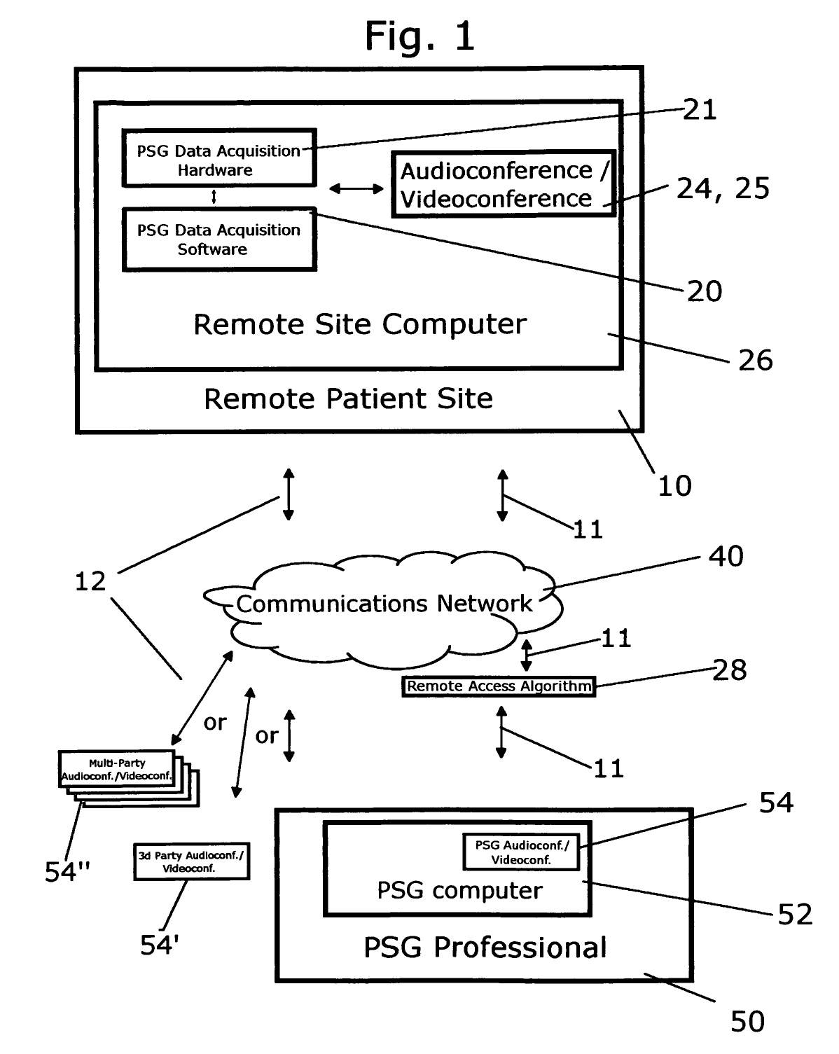

The preferred embodiments and best modes of the invention are shown in FIGS. 1 through 3. While the invention is described herein with regard to certain preferred embodiments, it is not intended that the present invention be so limited. On the contrary, it is intended to cover all alternatives, modifications, and equivalent arrangements as may be included within the spirit and scope of the invention as defined by the appended claims.

As polysomnography has become increasingly popular, portable polysomnography apparatuses that allow testing to be conducted in the patient's home or at other remote sites have become more prevalent. Until now, however, the availability of Type I polysomnography at these remote sites was limited as a polysomnography technician or other polysomnography professional was required to be physically nearby in order to monitor the patient. Type I PSG studies typically include periodic visual observations noted by the testing administrator. These observations may be impracticable with regard to procedures administered in the patient's home. However, known portable polysomnography apparatuses run on, are controlled by, mediated by, and/or otherwise accessed via well known computer operating systems, most commonly versions of Microsoft® Windows®, but may in addition include Apple® OS X®, versions of LINUX®, UNIX®, and the like. The present application recognizes that, with particular regard to systems mediated by a Windows® operating system (but equally applicable with regard to any other operating system), widely available additional hardware and or software may be combined to facilitate use of these systems remotely in order to administer Type I polysomnography. More specifically, hardware and/or software are available allowing the polysomnography apparatus to be accessed remotely, for example, via a remote access algorithm, graphical desktop sharing algorithm, and/or remote desktop application. Thereafter, the remote access algorithm, graphical desktop sharing algorithm, and/or remote desktop application may be used to mediate hardware and/or software to facilitate audioconferencing and/or videoconferencing between the patient and polysomnography administrator such that a Type I testing procedure may be conducted with the PSG professional “virtually” nearby, albeit physically distant from the patient and testing site. Alternatively, because the remote access algorithm, graphical desktop sharing algorithm, and/or remote desktop application and audioconferencing/videoconferencing functionality of the present invention are logically distinct and separate applications, the polysomnography administrator may elect to initiate audioconferencing/videoconferencing functionality between the patient and a third party, for example a treating physician. In yet another embodiment, multipoint audioconferencing/videoconferencing functionality may be utilized to interconnect an unlimited number of parties.

As shown schematically in FIG. 1, the Remote Patient Site 10 includes PSG data acquisition hardware 21, PSG data acquisition software 20, and remote site audioconferencing/videoconferencing hardware and/or software 24. This respective software is loaded onto remote site computer 26. The hardware is accessed and connected to, for example, the various PSG telemetry devices, a webcam or the like, a microphone or the like, and either external or integrated speakers. PSG data acquisition hardware 21 and associated software 20, for example but not limited to the Cleveland Medical Devices, Inc. (hereinafter “Cleveland Medical”) Sapphire® PSG apparatus and Crystal Monitor® software, is attached to the patient in a conventional manner.

The hardware 21 includes an electroencephalogram (EEG) which will generally use six “exploring” electrodes and two “reference” electrodes, unless a seizure disorder is suspected, in which case more electrodes will be applied to the patient to document the appearance of seizure activity. The exploring electrodes are usually attached to the scalp near the frontal, central (top) and occipital (back) portions of the brain via a paste that will conduct electrical signals originating from the neurons of the cortex. The readout from these electrodes provide indicia of brain activity that can be scored into different stages of sleep, for example, N1, N2, N3, or in combination, NREM sleep, REM sleep, and Wakefulness. An electrooculogram (EOG) utilizes two electrodes adjacent the right and left eyes. Changes measured in electropotential between the cornea and the retina (the cornea is positively charged relative to the retina) indicate the onset of REM sleep. Subsequently, onset of REM sleep facilitates determination of when sleep occurs. An electromyogram (EMG) typically includes four electrodes measuring muscle tension in the body and leg movements during sleep thereby providing indicia of, for example, periodic limb movement disorder, (PLMD). Two leads are placed on the chin with one above the jaw line and one below. Like the EOG described above, it facilitates the determination of sleep onset, particularly REM sleep. Sleep generally includes relaxation and hence a marked decrease in muscle tension occurs. A further decrease in skeletal muscle tension occurs in REM sleep. Additionally, because dreaming generally occurs during the REM stage, partial paralysis occurs in order to prevent the acting out of the dreams. Symptoms of REM behavior disorder include the failure of the partial paralysis to limit motion. Finally, two additional leads are placed on the anterior tibialis of each leg to further measure leg movements. Unlike the typical electrocardiogram (ECG or EKG) which utilizes ten electrodes, only two or three are utilized during polysomnography (PSG). These electrodes measure the electrical activity occurring in the beating heart muscle whereby the resulting waveforms may be analyzed for abnormalities indicating an underlying heart pathology. Nasal and oral airflow are measured using pressure transducers, and/or a thermocouple, fitted in or near the nostrils. This allows the clinician/researcher to measure rate of respiration and identify interruptions in breathing. Additionally plethysmographic methods are utilized to measure respiratory effort. Pulse oximetry indicates changes in blood oxygen saturation that often occur with sleep apnea and other respiratory problems. Finally, snoring may be recorded using an audio probe, although more commonly the sleep technician simply notes the occurrence of relevant snoring.

After attachment of the hardware to the patient, a remote access algorithm 28, for example but not limited to, the LogMeIn®, GoToMyPC®, GoToMeeting®, or Virtual Network Computing (VNC) service, is utilized to facilitate access to the remote site computer 26, which may be a laptop computer, via control channel 11 over a communications network 40, for example, the Internet, by a PSG professional 50 (typically at a central site) using a corresponding PSG computer 52 to administer the procedure. The PSG professional 50 is able to interface with and control the remote site computer 26 in order to “virtually” administer a polysomnographic examination or the like, to one or a group of patients, at any distance using the PSG computer 52. Moreover, the remote access algorithm 28 operating control channel 11 may be used to facilitate operation of PSG audioconferencing/videoconferencing hardware and/or software 54 loaded onto the PSG computer 52 operating via audio/video communications channel 12 whereby the PSG professional 50 is able to selectively visualize and/or conduct audio communications with the remote patient site 10 in order to conduct a Type I polysomnography procedure and any other video requirements. Control channel 11 and audio/video communications channel 12 are logically distinct but may operate on the same communications network 40, for example the Internet. Alternatively, the remote access algorithm 28 operating control channel 11 may be used to facilitate operation of third party audioconferencing/videoconferencing hardware and/or software 54′, wherein, for example, a treating physician is able to communicate with the Remote Patient Site 10 while the PSG Professional 50 is conducting testing. In yet another alternative, the remote access algorithm 28 operating control channel 11 may be used to facilitate operation of multipoint audioconferencing/videoconferencing hardware and/or software 54″, for example, the treating or referring physician and PSG Professional 50 are both able to communicate with the Remote Patient Site 10.

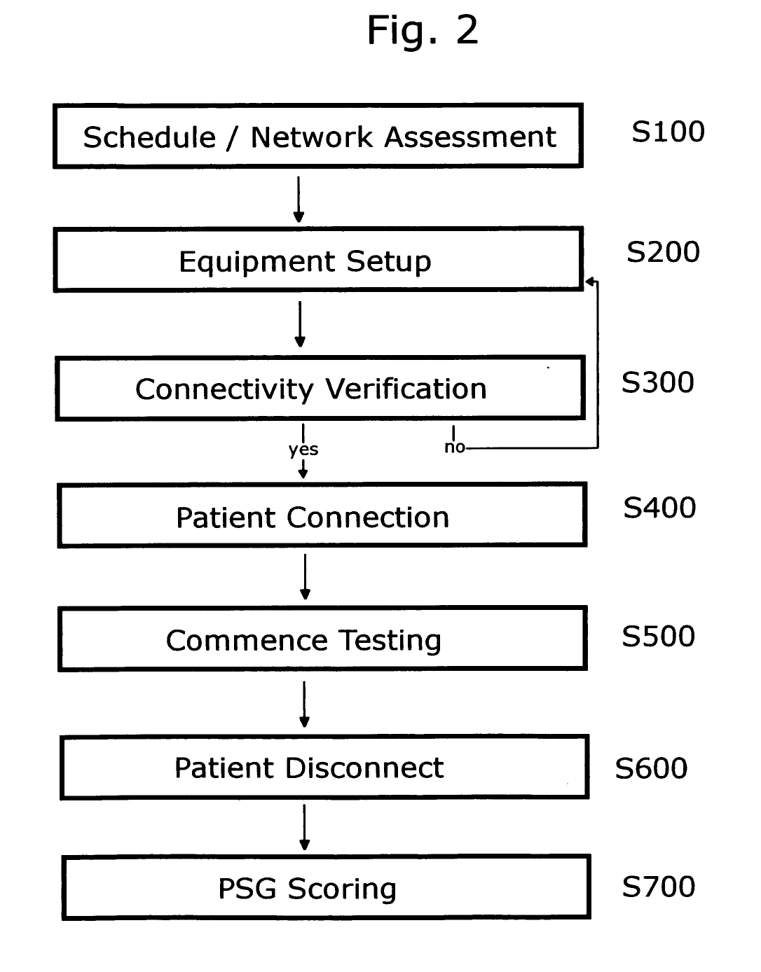

In an embodiment represented by the flowchart of FIG. 2, a Cleveland Medical Devices, Inc. Sapphire® PSG apparatus and Crystal Monitor® software is utilized in conjunction with its DreamPort® videoconferencing and video monitoring adjunct. A LogMeIn® remote computer support, management, and access suite of tools and the Skype® audioconferencing and videoconferencing service is used to facilitate the administration of Type I polysomnography by the polysomnography professional to a remote patient.

In FIG. 2, in an initial step S100, an appointment is scheduled with the patient, which may include utilizing a Internet-based medical practice management and reporting application, for example LeonardoMD®. Step S100 additionally includes an assessment of the availability of high speed Internet connectivity at the patient site, including wireless 3 G EVDO connectivity and commodity Internet access via cable modem, DSL, ISDN, fiber optic, or similar technology. Upon arrival of the setup technician at the appointed date and time, the equipment setup S200 commences with the remote patient site 10 (see FIG. 1) equipment 21 being connected and readied for use. As an example a Windows® operating system laptop computer 26 (see FIG. 1, remote site computer) running a PSG software suite, for example Cleveland Medical's Crystal Monitor® software is interconnected with its corresponding PSG telemetry devices, for example Cleveland Medical's Sapphire® PSG. Also loaded on the laptop computer 26 is audioconferencing/videoconferencing software and the necessary audio and video hardware required for functionality. It should be noted that the patient can optionally set up an appointment with an ENT physician for installation of a throat camera in the patient prior to the equipment setup S200.

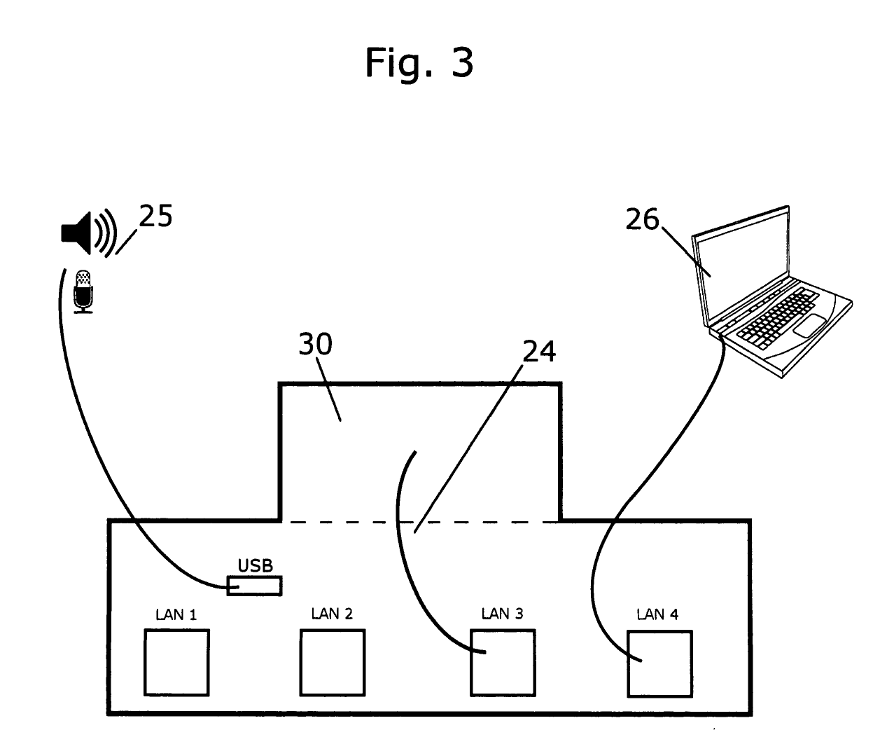

In the equipment setup, shown in schematic detail in FIG. 3, Skype® audioconferencing/videoconferencing software is utilized in conjunction with a Cleveland Medical DreamPort® video interface device 24 (see also FIG. 1, audioconference/videoconference) and a Polycom® audioconferencing combination speaker/microphone 25 (see also FIG. 1, audioconference/videoconference 24, 25). The “LAN4” port on the DreamPort® video interface device 24 is connected by crossover ethernet cable to the laptop computer 26. The attached DreamPort camera 30 is also connected to the “LAN3” port on the DreamPort® video interface device 24 and the Polycom® speaker/microphone 25 is connected to the USB port on the DreamPort® video interface device 24. In practice, the technician boots up the laptop computer 26 and ensures Internet connectivity via either: 1) a Verizon® UMW190 Air Card® utilizing the EVDO protocol or similar mobile telephony hi-speed Internet access network or 2) connects to the patient's home hi-speed Internet connection using an Asus® portable wireless router. In instances where the Asus® router is used, the technician will then utilize the laptop's integrated wireless connectivity to interface with the Asus® device. As seen in FIG. 2, Internet connectivity is then verified in step S300 by running the Skype® application on the laptop and ensuring that it is able to register online (as indicated by a green colored, check-marked icon). Skype® or a similar Internet-dependant application loaded on the remote site computer 26 is diagnostic regarding the availability of an Internet connection and hence the viability of conducting the procedure at the patient site 10. In instances where the technician is unable to log into Skype®, equipment setup S200 is repeated until a connection can be verified. Thereafter, patient connection S400 commences whereby the technician ensures that the DreamPort® video interface device 24 and its integrated DreamPort® camera 30 is positioned in order to best visualize the patient, typically at the foot of his/her bed. An ultraviolet illumination source (not shown) is also pointed in the general direction of the patient, being careful that the light itself is not visible through the DreamPort® camera 30. Ultraviolet illumination is invisible to the human eye and hence does not affect sleep, but is sufficient to illuminate the scene with regard to capturing video using the DreamPort® camera 30. The various leads of the Sapphire® PSG apparatus are then connected to the patient in a conventional manner. Prior to the technician leaving the house and if required by the testing protocal, the patient may be fitted with the paraphernalia required for continuous positive airway pressure (CPAP) measuring, for example, a mask and headgear interfacing with the Philips Respironics® REMstar® Auto CPAP device. In this example, the CPAP is connected to the DreamPort® via a serial port connection facilitating control of the CPAP using PC Direct® software residing on the DreamPort®. The monitoring technician is therefore able to remotely titrate pressure. The CPAP unit additionally includes a pressure transducer that allows oral nasal flow to be measured and recorded.

Testing may now commence in step S500 wherein a monitoring technician at a site removed from the Patient Site 10 (see FIG. 1) is able to access all of the equipment at the Patient Site 10 via the LogMeIn® or similar service, thereby enabling remote access and control of the Crystal Monitor® software interface on the Windows® laptop 26 (see FIG. 1, remote site computer). Bi-directional audio is enabled and the monitoring technician is able to visually monitor the movements of the patient via Skype® or the like. Preferably, bi-directional video may also be enabled via Skype® or the like. The monitoring technician is “virtually” nearby. He or she is able to manipulate the Cleveland Medical Crystal Monitor® software interface on the Windows® laptop, can see and hear the patient in order to conduct a Type I polysomnography procedure, and in addition can alert the patient in the event any apparatus becomes dislodged or otherwise requires adjustment. Because the patient and monitoring technician are able to see one another, the technician can, for example, demonstrate the proper placement of leads that require reattachment. Moreover, because the control channel 11 and audio/video communications channel 12 (see FIG. 1) are logically distinct, the monitoring technician may alternatively interconnect the Patient Site 10 to a third party audioconferencing/videoconferencing site 54′, for example, the treating physician while continuing to maintain control of the Remote Patient Site 10 via the control channel 11.

Upon completion of the testing and at a time pre-arranged with the patient, a technician returns to the Patient Site 10 to disconnect the patient as set forth in step S600. Thereafter, testing results are typically scored in step S700 in a conventional manner to determine whether the patient suffers from a sleep disorder and the degree of disability the patient is subject to.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What I claim is:

1) A method for remotely administering polysomnography comprising the steps of:
a) delivering and assembling polysomnography equipment at a patient site;
b) enabling remote access means to said assembled polysomnography equipment over a communications network via a control channel;
c) enabling real-time voice and video communication means over said communications network via a communications channel;
d) accessing said communications network;
e) interfacing said polysomnography equipment with a patient; and,
f) administering a polysomnography study on said patient from a remote central site.

2) A method for remotely administering polysomnography as claimed in claim 1 wherein said polysomnography study is a Type I polysomnography study.

3) A method for remotely administering polysomnography as claimed in claim 1 wherein said remote access means is a remote access algorithm means loaded on said polysomnographic equipment.

4) A method for remotely administering polysomnography as claimed in claim 1 wherein said real-time voice and video communication means is bi-directional real-time voice and video communication means.

5) A method for remotely administering polysomnography as claimed in claim 1 further comprising scoring said polysomnography study to determine a patient's sleep disability.

6) A method for remotely administering polysomnography as claimed in claim 1 wherein said communications network is the Internet.

7) A method for remotely administering polysomnography as claimed in claim 6 wherein said communications network is the Internet and is accessed via a technology selected from the group consisting of a mobile telephony hi-speed Internet access network, cable modem, DSL, ISDN, and fiber optic.

8) A method for remotely administering polysomnography as claimed in claim 1 wherein said control channel and said communications channel are logically distinct.

9) A method for remotely administering a Type I polysomnography study comprising the steps of:
a) delivering and assembling polysomnography equipment at a first patient site;
b) enabling remote access algorithm means to said polysomnography equipment over an Internet communications network via a control channel, said remote access algorithm means being loaded on said polysomnography equipment;
c) enabling real-time voice and video communication means over said Internet communications network via a communications channel;
d) accessing said Internet communications network;
e) interfacing said polysomnography equipment with a patient; and,
f) administering a Type I polysomnography study to said patient from a second site which is remote from said first patient site.

10) A method for remotely administering polysomnography as claimed in claim 9 wherein said real-time voice and video communication means is bi-directional real-time voice and video communication means.

11) A method for remotely administering polysomnography as claimed in claim 9 wherein said polysomnography equipment comprises polysomnography data acquisition hardware means and polysomnography data acquisition means.

12) A method for remotely administering polysomnography as claimed in claim 9 further comprising scoring said polysomnography study to determine a patient's sleep disability.

13) A method for remotely administering polysomnography as claimed in claim 9 wherein said Internet communications network is accessed via a technology selected from the group consisting of a mobile telephony hi-speed Internet access network, cable modem, DSL, ISDN, and fiber optic.

14) A method for remotely administering polysomnography as claimed in claim 9 wherein said control channel and said communications channel are logically distinct.

15) A method for remotely administering polysomnography to diagnose sleep apnea comprising the steps of:
a) delivering and assembling polysomnography equipment at a first site;
b) enabling remote access algorithm means to said polysomnography equipment over the Internet via a control channel;
c) enabling real-time voice and video communication means over said Internet via a communications channel;
d) accessing said Internet;
e) interfacing said polysomnography equipment at said first site with a patient; and,
f) administering a Type I polysomnography study to said patient from a second site which is remote from said first site.

16) A method for remotely administering polysomnography to diagnose sleep apnea as claimed in claim 15 wherein said real-time voice and video communication means are accessible by a third party.

17) A method for remotely administering polysomnography to diagnose sleep apnea as claimed in claim 15 wherein said real-time voice and video communication means are accessible by a plurality of parties.

18) A method for remotely administering polysomnography comprising the steps of:
a) delivering and assembling polysomnography hardware means and polysomnography software means to a plurality of patient sites;
b) enabling remote access algorithm means to said polysomnography hardware and software means at each patient site over a communications network via a control channel, said remote access algorithm means being loaded on said polysomnography hardware and software means;
c) enabling real-time voice and video communication means over said communications network via a communications channel;
d) accessing said communications network;
e) interfacing said polysomnography hardware and polysomnography software means with a patient; and,
f) administering a polysomnography study to each of said patients from a second site which is remote from each patient site.

19) A method for remotely administering polysomnography as claimed in claim 18 wherein said second site is a central second site able to administer multiple polysomnography studies essentially simultaneously.

20) A method for remotely administering polysomnography as claimed in claim 18 wherein said polysomnography study is a Type I polysomnography study.
